(19) United States
(12) Patent Application Publication
(10) Pub. No.: US 2022/0160628 A1
(43) Pub. Date: May 26, 2022

(54) PROCESS FOR MANUFACTURING IMPROVED SEMI-SOFT SOLID DOSAGE FORMS AND SOFT CHEWS PRODUCED THEREBY

(71) Applicant: FLORIDA RESEARCH GROUP LLC, Tampa, FL (US)

(72) Inventor: KOTHA SEKHARAM, TAMPA, FL (US)

(21) Appl. No.: 17/534,947

(22) Filed: Nov. 24, 2021

Related U.S. Application Data
(60) Provisional application No. 63/117,823, filed on Nov. 24, 2020.

(51) Int. Cl. A61K 9/00; A61K 47/36; A61K 47/38; A61K 47/10; A61K 47/24; A61K 47/34; A61K 47/46; A61K 47/26; A61K 47/12; A23K 20/163; A23K 20/105; A23K 20/158; A23K 10/20; A23K 40/25; A23L 33/125; A23L 33/12; A23P 30/20

(52) U.S. Cl. CPC A61K 9/0056; A23V 2002/00; A61K 47/38; A61K 47/10; A61K 47/24; A61K 47/34; A61K 47/46; A61K 47/26; A61K 47/12; A23K 20/163; A23K 20/105; A23K 20/158; A23K 10/20; A23K 40/25; A23L 33/125; A23L 33/12; A23P 30/20; A61K 47/36

(57) ABSTRACT

The invention relates to semi-soft dosage forms with functional health ingredients for animal as well as human use.

### CROSS REFERENCE TO A RELATED APPLICATION

This application claims the benefit of U.S. Provisional Application Ser. No. 63/117,823, filed Nov. 24, 2020, which is incorporated herein by reference in its entirety.

### BACKGROUND OF THE INVENTION

Functional health ingredients are delivered to end users in a variety of different ways; solid dosages are the most common forms. Tablets, chewable tablets, orally disintegrating tablets, capsules, and powders are some examples of solid oral dosages. Out of all currently available forms, soft chews are gaining more attention because of the bite, texture, chewiness, taste flavor and overall mouthfeel.

Soft chews are generally made by combining active ingredients with excipients, binders, humectants, oils, sweeteners, flavors, colors and preservatives to make a dough and then shaping the dough to desired shape, size and weight.

If the composition is not appropriate, soft chews become hard and fail to perform their intended purpose. Also, if the composition is not proper, it may affect the stability of certain active ingredients. For example, the presence of water may affect moisture-sensitive functional ingredients such as acetyl salicylic acid. Further, if the process temperature is high, it may affect thermo-labile active ingredients; if the composition includes oxidation-triggering ingredients, it may affect stability of oxidation-prone active ingredients; high water activity may affect enzyme activity as well as viability of probiotics.

Having so many factors affecting finished forms, there is always a need for improved compositions to make semi solid dosages.

Edwin Christensen (WO2006036552A2) reported making nutritional soft chew product with 10% to about 50% by weight of a starch component, about 25% to about 50% by weight of a sugar component, about 0-20% of a humectant component, and about 5% to about 25% by weight of water.

U.S. Pat. No. 8,114,455B2 (Neil E. Paulsen et al) teaches process for manufacturing chewable dosage forms for drug delivery with active ingredient, inactive ingredient, non-aqueous fluid; wherein the non-aqueous fluid is an oil such as corn, safflower, cottonseed, soybean and olive oils. Edible soft chew formulation, Example-1 lists vegetable oil (soy bean oil) at 7% of the formulation.

U.S. Pat. No. 8,865,240B2 (Neil E. Paulsen et al) teaches a process for manufacturing an edible soft chewable medication comprising blending an active ingredient with an oil and dry inactive ingredients including a flavoring, a binder, a bulking agent, a humectant and an excipient.

Usage of vegetable oils as inactive ingredients may cause oxidative degradation of certain active ingredients up on storage and at higher temperatures. In addition, oil may seep out of chews or segregate on storage. Since active ingredients are blended with oil, content uniformity may become a problem.

Also, all fats and oils are prone to oxidation. Rapidity of the oxidation depends on the degree of unsaturation of the fatty acids in the fat or oil and also the presence to antioxidants.

Other external factors may influence lipid oxidation is high temperature, oxygen and light. Oxidative Stability Index (OSI) is a method that determines the relative resistance of fats or oils to oxidation.

Higher OSI values are better for any product. OSI values of some of the oils and fats are as follows: fish oil (0.5 to 2.5 h at 110° C.), sunflower oil (3.0 to 6.0 h), soybean oil (5.0 to 6.7 h), canola oil (6.0 to 8.0 h), corn oil (7.0 to 11.0 h), pork fat-lard (18 h), beef tallow (69 h), partially hydrogenated soybean oil (20.0 to 85.0 h).

EP2922526A1 (Dominique Kluger et al) teaches manufacturing of semi-plastic pharmaceutical dosage units by mixing at least one active pharmaceutical ingredient with one or more dry and/or liquid components and by heating a forming agent until melting to faun a dough, and shaping, wherein the temperature of the dough is between 35° C. and 45° C. According to the process described, a defined amount of the molten PEG was added quickly to the chew mixture in the horizontal plough share mixing blender, which is a commercially challenging process. This mixture was then mixed until homogeneous and could be separated from the wall. The temperature of the resulting dough was stated as 43° C. The mixture resembled a “cookie dough-like” appearance.

Temperature of the dough may be critical for the stability of certain heat-labile active ingredients. There is a need to minimize the dough temperature to less than 43° C. stated in EP2922526A1 (Dominique Kluger et al)

One common ingredient in almost all patents is the usage of polyethylene glycol (PEG) as an anti-sticking agent. This is acceptable for making soft chews for animals. However, PEG imparts unacceptable bitter taste and hence not suitable to use in chews for humans.

### BRIEF SUMMARY OF THE INVENTION

In accordance with the subject invention, it has been discovered that palatable soft chews can be made without using PEG. Thus, one aspect of the invention provides palatable semi-soft solid dosage form compositions that do not contain PEG.

Another aspect of the invention provides a semi-soft solid dosage form composition that does not contain an oil as an inactive ingredient.

In another aspect, the subject invention provides a process for manufacturing improved semi-soft solid dosage form comprising:

a) blending an active ingredient with one or more excipients and/or wetting agents;

b) adding solid fat melted to at least 40° C. along with one or more release agents;

c) blending these ingredients to form a dough at a temperature below 35° C., and

d) shaping such formed dough to a desired shape, weight, size and hardness that can be packaged as an individual dosage.

Advantageously, the process according to the present invention allows manufacture with or without PEG at low temperatures.

The excipient can be selected from, for example, starch, cellulose, flour, sorbitol, sucrose, mannitol, other sugars, brewer's dried yeast, calcium carbonate, dicalcium phosphate, or a combination thereof. In a preferred embodiment, the excipient is starch. The excipient may comprise, for example, 5% to 75%, 10% to 60%, 20% to 50%, or 30% to 40% of the edible soft chew.

The active ingredient can be selected from, for example, anthelmetics, analgesics, antibiotics, antivirals, anti-inflammatories, endoparasiticides, ectoparasiticides, antifungals, sedatives, anxiolytics, health supplements, therapeutic agents, analgesics, antacids, antianxiety drugs, antiarrhythmics, antibacterials, antibiotics, anticoagulants, anticonvulsants, antidepressants, antidiarrheals, antiemetics, antifungals, antihistamines, antihypertensives, anti-inflammatories, antineoplastics, antipsychotics, antipyretics, antivirals, barbiturates, beta-blockers, bronchodilators, cold cures, corticosteroids, cough suppressants, cytotoxics, decongestants, diuretics, expectorants, hormones, hypoglycemics (oral), immunosuppressives, laxatives, muscle relaxants, sedatives, sex hormones, sleep inducers, tranquilizers, vitamins, and combinations thereof.

The wetting agent can be selected from, for example, glycerin, polyethylene glycol, propylene glycol, and combinations thereof. In preferred embodiments, the wetting agent comprises about 5% to about 30% (w/w) of the soft chew.

The release agent can be selected from, for example, lecithin, PEG, gum arabic and combinations thereof. In preferred embodiments, the release agent constitutes from about 1 to about 10 percent (w/w) of the edible soft chew. In one embodiment, PEG is not used.

The solid fat can be selected from, for example, vegetable shortening, beef tallow, pork fat, stearic acid, magnesium stearate, and combinations thereof. Preferably, the solid fat comprises fat that remains solid at 20° C. and has a melting point of 30° C. or above. In a preferred embodiment, the fat constitutes from about 1 to about 15% (w/w) of the soft chew.

Optional ingredients that can be used in the process of the subject invention include, for example, flavorings, colorings, palatants, and/or sweeteners.

The subject invention further provides soft chews made by the method described herein.

### DETAILED DESCRIPTION OF THE INVENTION

In accordance with the subject invention, it has been discovered that palatable soft chews can be made without using PEG. Thus, one aspect of the invention provides palatable semi-soft solid dosage form compositions that do not contain PEG.

Another aspect of the invention provides a semi-soft solid dosage form composition that does not contain an oil as an inactive ingredient.

In another aspect, the subject invention provides a process for manufacturing improved semi-soft solid dosage form comprising:

a) blending an active ingredient with one or more excipients and/or wetting agents;

b) adding solid fat melted to at least 40° C. along with one or more release agents;

c) blending these ingredients to form a dough at a temperature below 35° C., and

d) shaping such formed dough to a desired shape, weight, size and hardness that can be packaged as an individual dosage.

Advantageously, the process according to the present invention allows manufacture with or without PEG at low temperatures. Therefore, the process allows versatility in formulations whenever it is desirable to exclude PEG from the semi-soft solid dosage form compositions, such as when a bitter taste imparted by PEG is not desirable in a composition for human use.

The excipient can be selected from, for example, starch, cellulose, flour, sorbitol, sucrose, mannitol, other sugars, brewer's dried yeast, calcium carbonate, dicalcium phosphate, or a combination thereof. In a preferred embodiment, the excipient is starch. The excipient may comprise, for example, 5% to 75%, 10% to 60%, 20% to 50%, or 30% to 40% of the edible soft chew.

The active ingredient can be selected from, for example, anthelmetics, analgesics, antibiotics, antivirals, anti-inflammatories, endoparasiticides, ectoparasiticides, antifungals, sedatives, anxiolytics, health supplements, therapeutic agents, analgesics, antacids, antianxiety drugs, antiarrhythmics, antibacterials, antibiotics, anticoagulants, anticonvulsants, antidepressants, antidiarrheals, antiemetics, anti fungals, antihistamines, antihypertensives, anti-inflammatories, antineoplastics, antipsychotics, antipyretics, anti virals, barbiturates, beta-blockers, bronchodilators, cold cures, corticosteroids, cough suppressants, cytotoxics, decongestants, diuretics, expectorants, hormones, hypoglycemics (oral), immunosuppressives, laxatives, muscle relaxants, sedatives, sex hormones, sleep inducers, tranquilizers, vitamins, and combinations thereof.

The wetting agent can be selected from, for example, glycerin, polyethylene glycol, propylene glycol, and combinations thereof. In preferred embodiments, the wetting agent comprises about 5% to about 30% (w/w) of the soft chew.

The release agent can be selected from, for example, lecithin, PEG, gum arabic and combinations thereof. In preferred embodiments, the release agent constitutes from about 1 to about 10 percent (w/w) of the edible soft chew. In one embodiment, PEG is not used.

The solid fat can be selected from, for example, vegetable shortening, beef tallow, pork fat, stearic acid, magnesium stearate, and combinations thereof. Preferably, the solid fat comprises fat that remains solid at 20° C. and has a melting point of 30° C. or above. In a preferred embodiment, the fat constitutes from about 1 to about 15% (w/w) of the soft chew.

Optional ingredients that can be used in the process of the subject invention include, for example, flavorings, colorings, palatants, and/or sweeteners.

In one embodiment, gelatin is used as a binding agent. Gelatin is obtained by the partial hydrolysis of collagen derived from, for example, skin, white connective tissues, and bones of animals. Gelatin is nearly tasteless and odorless, is a vitreous, brittle solid that is faintly yellow in color. Gelatin is soluble in aqueous solutions of polyhydric alcohols such as glycerol and propylene glycol.

In certain embodiments, the active agent is a pharmaceutical or nutraceutical agent. The active agent can also be a microbial culture, for example, a probiotic culture.

Non-limiting examples of the active agents include analgesics, anti-inflammatories, minerals, nutraceuticals, antibiotics, antivirals, endoparasiticides, ectoparasiticides, anthelmetics, antifungals, probiotics, vitamins, herbs, and combinations thereof.

Preferred active agents include: SAMe, Co enzyme Q-10, omega oils, fish oil, Perna canaliculus, palmitoylethanolamide, ivermectin, amoxicillin, carprofen, firocoxib, enrofloxacin, lindamycin, and acetyl alicylic acid.

Preferred probiotics include: Lactobacillus acidophilus, Bifidobacterium thermophilum, B. longum, L. fermentum, L. casei, B. bifidum, E. faecium, and B. coagulans.

Other active agents include: non-steroidal anti-inflammatory drugs (e.g, carprofen, flunixine meglumine, ketoprofen, ketoprofen methyl ester, naproxen, meloxicam, robenacoxib, and firocoxib), medetomidine, phenylbutazone, hydromorphone, anti-emetics (e.g., maropitant, and maropitant salts, dextromethorphan, diphenhydramine, 8-chlorotheophylline, cisapride, omeprazol, famotidine, metoclopramide, promethazine, dolasetron, ondansetron, granisetron, ketamine, lansoprasol, meclizine, and mirtazepine), antihistamines/antipyretics (e.g., acepromazine, clemastine fumarate, cyproheptadine, famotidine, loratadine, hydroxyzine, meclizine hydrochloride, apoquel, chlorpheniramine, and diphenhydramine), antiparasitics (e.g., macrocyclic lactones (ivermectin, abamectin, doramectin, emamectin, moxidectin, milbemycin, and milbemycin oxime), imidacloprid, emodepside, levamisole, pyrantel, pyrantel pamoate, isoxazolines (e.g., sarolaner, afoxolaner, lotilaner, and fluralaner), derquantel, anticoccidials, benzimidazoles (thiabendazole, mebendazole, fenbendazole, oxfendazole, and albendazole), antimicrobials (e.g., pleuromutilins, polymyxins, aminoglycosides, fluoroquinolones (e.g., danofloxacin, ciprofloxacin, norfloxacin, ofloxacin, and levofloxacin), macrolides (e.g., azithromycin, erythromycin, and telithromycin), lincosamides (e.g., clindamycin), aminoglycosides (e.g., amikacin, streptomycin, and tobramycin), sulfonamides (e.g., sulfadoxine, sulfamethizole, and sulfisoxazole), penicillins, beta-lactams, tetracyclines (e.g., doxycycline hyclate, and minocycline), aminopenicillins, cephalosporins (1^{st}-4th generations, e.g., simplicef, ceftiofur, and cefovecin), prednisolone, and methylprednisolone.

Combinations of one or more active agents are also envisioned. Additional active agents are known to a person of ordinary skill in the art and such embodiments are within the purview of the invention.

The amount of an active agent included in the soft chews produced according to the methods of the invention ranges from about 0.1% to about 25% w/w of the soft chew. A therapeutically effective amount of an active agent may vary depending upon the intended application, the subject, and the condition being treated, the weight and age of the subject, and the severity of the condition. These parameters can readily be determined by one of ordinary skill in the art.

The subject invention further provides soft chews made by the method described herein.

Optionally, soft chews of the invention may include additional ingredients commonly used in compositions for humans and animal use. For example, flavoring agents such as molasses, carrot, apple, bacon, hickory flavor; coloring agents such as iron oxide, and titanium dioxide; sweeteners such as sugar, dextrose, sodium saccharin, and sucralose; preservatives such as parabens, propionate, and sorbic acid; and antioxidants such as hydroxyanisole (BHA), butylated hydroxytoluene (BHT), and tertiary-butyl hydroquinone (TBHQ) can also be added. These ingredients can be added in any of the several mixing steps in the methods of the invention described above.

Soft chews of the invention can be used to orally deliver an active agent to a subject. Accordingly, methods of orally delivering an active agent to a subject in the form of soft chews of the invention are also provided.

The subject can be a human or a non-human animal, preferably, a mammal. When the subject is a human, the subject can be, for example, a child or a senior. When the subject is a non-human animal, the subject can be, for example, a domestic pet, such as a dog or a cat. The subject can also be, for example, a horse, cow, pig, camel, or ferret.

### Definitions

As used herein, the singular forms “a”, “an” and “the” are intended to include the plural forms as well, unless the context clearly indicates otherwise. Further, to the extent that the terms “including”, “includes”, “having”, “has”, “with”, or variants thereof are used in either the detailed description and/or the claims, such terms are intended to be inclusive in a manner similar to the term “comprising.”

The transitional term “comprising,” which is synonymous with “including,” or “containing,” is inclusive or open-ended and does not exclude additional, un-recited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention. Use of the term “comprising” contemplates other embodiments that “consist” or “consist essentially of” the recited component(s).

The term “about” means within an acceptable error range for the particular value as determined by one of ordinary skill in the art, which will depend in part on how the value is measured or determined, i.e., the limitations of the measurement system. Where particular values are described in the application and claims, unless otherwise stated the term “about” meaning within an acceptable error range for the particular value should be assumed. In the context of compositions containing amounts of ingredients where the term “about” is used, these compositions contain the stated amount of the ingredient with a variation (error range) of 0-10% around the value (X±10%). Where the term “about” is used to describe target temperatures or durations of time used in certain processes, the target temperatures of durations of time can be varied within a range 0-10% around the target value (X±10%).

The term “w/w” as used herein to describe an amount of an ingredient in the soft chews of the subject invention indicates that the corresponding ingredient is present in the specified weight ratio of the ingredient to the total weight of the soft chew.

In the present disclosure, ranges are stated in shorthand, to avoid having to set out at length and describe each and every value within the range. Any appropriate value within the range can be selected, where appropriate, as the upper value, lower value, or the terminus of the range. For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, and 0.7-1.0.

When ranges are used herein, such as for dose ranges, combinations and sub-combinations of ranges (e.g., subranges within the disclosed range), specific embodiments therein are intended to be explicitly included.

A “soft chew” as used herein refers to a pharmaceutical unit dose that is solid at room temperature and has a rubbery texture appropriate for mastication in the mouth. Soft chew is particularly suitable for administration of an active agent to a non-human animal, such as pets, because non-human animals do not prefer swallowing pills. Indeed, administering a solid pill to a non-human animal is difficult because of the resistance the non-human animal may provide to such administration. Non-human animals prefer eating a soft chew because the non-human animals can experience and enjoy flavor that can be incorporated in the soft chew. Such soft chews have a softness that is similar to a cooked ground meat patty.

A greater understanding of the present invention and of its many advantages may be had from the following examples, given by way of illustration. They are not to be considered as limiting the invention.

### Example:1

An example of improved semi-solid dosage for animal use is set forth below.

Formula-1

| | W/W |
|---|---|
| | % |
| Active ingredient | 2.7 |
| Potato starch | 43.3 |
| Croscarmellose sodium | 10.0 |
| Glycerin | 20.0 |
| Lecithin | 2.0 |
| PEG 3350 | 8.0 |
| Beef tallow | 12.0 |
| Flavor | 2.0 |

All powdered ingredients (Active ingredient, potato starch, croscarmellose sodium) are weighed and charged to dough mixer and blended for 5 minutes. Glycerin and flavor are weighed, added to the blender and blended for 2 minutes. Lecithin, PEG 3350 and beef tallow are weighed and heated together to melt and such hot mix at 40° C. is added to the blender. Contents are blended for about 5 minutes to form a smooth shapeable dough. Finished dough has a temperature less than 35° C. The dough is passed through any customary shaping device, including an extruder to form chews of right shape and uniform weights. Chews are cured for 24 hours and packaged.

This example establishes that semi-soft chews can made (1) without using any oil in the composition and (2) at reduced dough temperature.

### Example:2

An example of improved semi-solid dosage for human use is set forth below.

Formula

| | W/W % |
|---|---|
| Active ingredient | 2.7 |
| Sorbitol | 33.3 |
| Croscarmellose sodium | 10.0 |
| Wheat flour | 17.3 |
| Glycerin | 10.7 |
| Lecithin | 2.0 |
| Gum arabic | 8.0 |
| Shortening | 12.0 |
| Stearic acid | 2.0 |
| Flavor | 2.0 |

All powdered ingredients are weighed and charged to dough mixer and blended for 5 minutes. Glycerin and flavor are weighed, added to the blender and blended for 2 minutes. Lecithin, gum arabic, stearic acid and shortening are weighed and heated together to melt and such hot mix having a temperature of 40° C. is added to ingredients in the blender. Contents are blended for about 5 minutes to form a smooth shapeable dough. Finished dough has a temperature less than 35° C. The dough is passed through any customary shaping device, including an extruder to form chews of right shape and uniform weights. Chews are cured for 24 hours and packaged.

This example shows that semi-soft chews can made (1) without a need of PEG, yet achieving desired hardness and ease of handling the dough, (2) avoiding bitter off taste of the finished product, and (3) at reduced dough temperature.

I claim:

1. A process for manufacturing improved semi-soft solid dosage comprising:
a) blending an active ingredient with one or more excipients and/or wetting agents;
b) adding solid fat melted to at least 40° C. along with one or more release agents;
c) blending these ingredients to form a dough at a temperature below 35° C.; and
d) shaping the formed dough to a desired shape, weight, size and hardness that can be packaged as an individual dosage.

2. The process of claim 1, wherein the excipient is selected from the group consisting of starch, cellulose, flour, sorbitol, sucrose, mannitol, sugars, brewers dried yeast, calcium carbonate, dicalcium phosphate, sugar, or a combination thereof.

3. The process of claim 2, where in the excipient constitutes from about 10 to about 60 percent (w/w) of the edible soft chew

4. The process of claim 1, where in the excipient comprises starch.

5. The process of claim 1, wherein the active ingredient is selected from the group consisting of anthelmetics, analgesics, antibiotics, antivirals, anti-inflammatories, endoparasiticides, ectoparasiticides, antifungals, sedatives, anxiolytics, health supplements, therapeutic agents, analgesics, antacids, antianxiety drugs, antiarrhythmics, antibacterials, antibiotics, anticoagulants, anticonvulsants, antidepressants, antidiarrheals, antiemetics, antifungals, antihistamines, antihypertensives, anti-inflammatories, antineoplastics, antipsychotics, antipyretics, antivirals, barbiturates, beta-blockers, bronchodilators, cold cures, corticosteroids, cough suppressants, cytotoxics, decongestants, diuretics, expectorants, hormones, hypoglycemics (oral), immunosuppressives, laxatives, muscle relaxants, sedatives, sex hormones, sleep inducers, tranquilizers, vitamins, and combinations thereof.

6. The process of claim 1, wherein the wetting agent is selected from the group consisting of glycerin, polyethylene glycol, propylene glycol, and combinations thereof.

7. The process of claim 6, wherein the wetting agent is glycerin.

8. The process of claim 6, wherein the wetting agent constitutes from about 5 to about 30 percent (w/w) of the edible soft chew

9. The process of claim 1, wherein the release agent is selected from the group consisting of lecithin, PEG, gum arabic and combinations thereof.

10. The process of claim 9, wherein the release constitutes from about 1 to about 10 percent (w/w) of the edible soft chew.

11. The process of claim 1, wherein the solid fat is selected from the group consisting of vegetable shortening, beef tallow, pork fat, stearic acid, magnesium stearate, and combinations thereof.

12. The process of claim 11, wherein the solid fat comprises solid fat that remains solid at 20° C. and has melting point of 30° C. or above.

13. The process of claim 11, wherein the fat constitutes from about 1 to about 15% (w/w) of the edible soft chew.

14. The process of claim 1, wherein optional ingredients include one or more flavorings, colorings, palatants, and/or sweeteners.

15. A soft chew produced by the process of claim 1.
